(19) United States
(12) Patent Application Publication
(10) Pub. No.: US 2014/0031934 A1
(43) Pub. Date: Jan. 30, 2014

(54) SACRO-ILIAC JOINT IMPLANT SYSTEM AND METHOD

(71) Applicant: Hai H. Trieu, Cordova, TN (US)

(72) Inventor: Hai H. Trieu, Cordova, TN (US)

(73) Assignee: WARSAW ORTHOPEDIC, INC., Warsaw, IN (US)

(21) Appl. No.: 13/558,900

(22) Filed: Jul. 26, 2012

(51) Int. Cl. A61F 2/44

(52) U.S. Cl. 623/17.11

(57) ABSTRACT

A sacro-iliac implant includes an inner member having an inner surface and an outer surface. The inner member extends between a first end and a second end configured for penetrating a sacrum. An outer member extends between a first end including a flange and being configured to engage an outer non-articular surface of an ilium and a second end. The inner member is rotatable relative to the outer member such that the outer surface of the inner member adjacent its first end engages the outer member to cause axial translation of the inner member relative to the outer member in a configuration such that separated articular surfaces of the sacrum and the ilium are drawn into fixation. Systems and methods of use are disclosed.

### TECHNICAL FIELD

The present disclosure generally relates to medical devices for the treatment of musculoskeletal disorders, and more particularly to an implant system and method for treating the sacro-iliac joint.

### BACKGROUND

The sacroiliac (SI) joint is a diarthrodial joint that joins the sacrum to the ilium bones of the pelvis. In the SI joint, the sacral surface has hyaline cartilage that moves against fibrocartilage of the iliac surface. The spinal column is configured so that the weight of an upper body rests on the SI joints at the juncture of the sacrum and ilia. Stress placed on the SI joints in an upright position of the body makes the lower back susceptible to injury.

Disorders of the SI joint can cause low back and radiating buttock and leg pain in patients suffering from degeneration and laxity of the SI joint. In some cases, the SI joint can undergo dehydration and destabilization, similar to other cartilaginous joints, which causes significant pain. The SI joint is also susceptible to trauma and degeneration, from fracture and instability. It is estimated that disorders of the SI joint are a source of pain for millions of people suffering from back and radicular symptoms.

Non-surgical treatments, such as medication, injection, mobilization, rehabilitation and exercise can be effective, however, may fail to relieve the symptoms associated with these disorders. Surgical treatment of these disorders includes stabilization and/or arthrodesis procedures, which may employ fixation devices. Arthrodesis may include immobilization of a joint. The present disclosure describes an improvement over these prior art technologies.

### SUMMARY

Accordingly, an implant system and method is provided for treating the SI joint. It is contemplated that the system may include an implant configured for disposal with the SI joint. It is further contemplated that the implant system and method may be employed for an arthrodesis treatment.

In one particular embodiment, in accordance with the principles of the present disclosure, a sacro-iliac implant is provided. The sacro-iliac implant includes an inner member having an inner surface and an outer surface. The inner member extends between a first end and a second end configured for penetrating a sacrum. An outer member extends between a first end including a flange and being configured to engage an outer non-articular surface of an ilium and a second end. The inner member is rotatable relative to the outer member such that the outer surface of the inner member adjacent its first end engages the outer member to cause axial translation of the inner member relative to the outer member in a configuration such that separated articular surfaces of the sacrum and the ilium are drawn into fixation.

In one embodiment, the sacro-iliac implant includes an inner member including an inner surface and extending between a first end and a second end configured for penetrating a sacrum. An outer member extends between a first end configured to engage an outer non-articular surface of an ilium and a second end. An actuator is disposed with the outer member and defines an outer surface. The actuator is movable relative to the outer member such that the outer surface of the actuator engages the inner surface to cause axial translation of the inner member relative to the outer member in a configuration such that separated articular surfaces of the sacrum and the ilium are drawn into fixation.

### BRIEF DESCRIPTION OF THE DRAWINGS

The present disclosure will become more readily apparent from the specific description accompanied by the following drawings, in which:

FIG. 1 is a side view of one particular embodiment of an implant system in accordance with the principles of the present disclosure;

FIG. 2 is a side, cross section view of the implant system shown in FIG. 1;

FIG. 3 is a plan view, in part cross section of one particular embodiment of an implant system in accordance with the principles of the present disclosure disposed with the sacro-iliac region;

FIG. 4 is a plan view, in part cross section of one particular embodiment of an implant system in accordance with the principles of the present disclosure disposed with the sacro-iliac region;

FIG. 5 is a side view of one particular embodiment of an implant system in accordance with the principles of the present disclosure;

FIG. 6 is a side, cross section view of the implant system shown in FIG. 5;

FIG. 7 is a side view of one particular embodiment of an implant system in accordance with the principles of the present disclosure;

FIG. 8 is a perspective, cross section view of the implant system shown in FIG. 7;

FIG. 9 is a perspective view of the implant system shown in FIG. 7;

FIG. 10 is an end view of the implant system shown in FIG. 7;

FIG. 11 is a perspective view of a component of the implant system shown in FIG. 7;

FIG. 12 is a perspective view of the component shown in FIG. 7;

FIG. 13 is a side view of one particular embodiment of an implant system in accordance with the principles of the present disclosure;

FIG. 14 is a perspective view of the implant system shown in FIG. 13;

FIG. 15 is a perspective view of a component of the implant system shown in FIG. 13;

FIG. 16 is a side, cross section view of the component shown in FIG. 15;

FIG. 17 is a perspective, cross section view of a component of the implant system shown in FIG. 13; and

FIG. 18 is a perspective view of the component shown in FIG. 17.

Like reference numerals indicate similar parts throughout the figures.

### DETAILED DESCRIPTION

The exemplary embodiments of the surgical system and methods of use disclosed are discussed in terms of medical devices for the treatment of musculoskeletal disorders and more particularly, in terms of a surgical system and method for treating the sacro-iliac (SI) joint. It is envisioned that the surgical system and methods of use disclosed provide stability and maintains structural integrity while reducing stress on the SI joint. It is further envisioned that the present disclosure may be employed to treat musculoskeletal disorders including SI dysfunction or syndrome, dehydration, destabilization and/or laxity.

In one embodiment, the system includes a screw having a lag configuration for SI joint fusion with compression capability. The screw comprises a distal thread for engaging sacral bone and a non-threaded proximal section enables compression. In one embodiment, the non-threaded proximal section has a round cross-section. In one embodiment, the screw defines a large screw head for increased compression capability. A drive screw mechanism is incorporated into the screw head. It is contemplated that the screw head defines a hex, star or square drive insert. In one embodiment, the system includes a reinforcing sleeve or a screw having a larger inner diameter to strengthen shear strength at the proximal section. In one embodiment, the system includes a screw for SI joint fusion with adjustable length and additional compression capability. In one embodiment, the length of the screw is adjustable and the screw is rotationally and linearly compressible.

In one embodiment, the system comprises a cannulated compression screw assembly and method of use to stabilize the SI joint for fusion. In one embodiment, the screw assembly includes an anchoring section, reinforcement section, compression section and a compression locking nut/bolt. The screw assembly is configured to provide stability across SI joint. In one embodiment, the system comprises a method comprising the steps of: under image guidance such as fluoroscopy, inserting a guidewire across the SI joint at a trajectory; drilling and tapping across the SI joint along the guidewire; with the guidewire in place, inserting the anchoring section of the screw assembly through the ilium into the sacrum until appropriate depth is reached; and advancing the reinforcement section across the SI joint while compressing the SI joint using the proximal compression nut/bolt. The surface of the screw assembly may be treated such that it would enhance osseointegration (for example, textured, anodized, HA-coated and/or porous coating).

In one embodiment, the screw assembly may be disposed on one or more materials such as titanium, titanium alloys, CoCr alloys, stainless steel, PEEK and/or carbon-reinforced PEEK. In one embodiment, the reinforcement section may have round or non-rounded shapes such as square, triangle, hexagonal and/or star. In one embodiment, the screw assembly can be implanted without the use of guidewire. In one embodiment, a cannulation channel can be used for injection of biological or pharmacological agents.

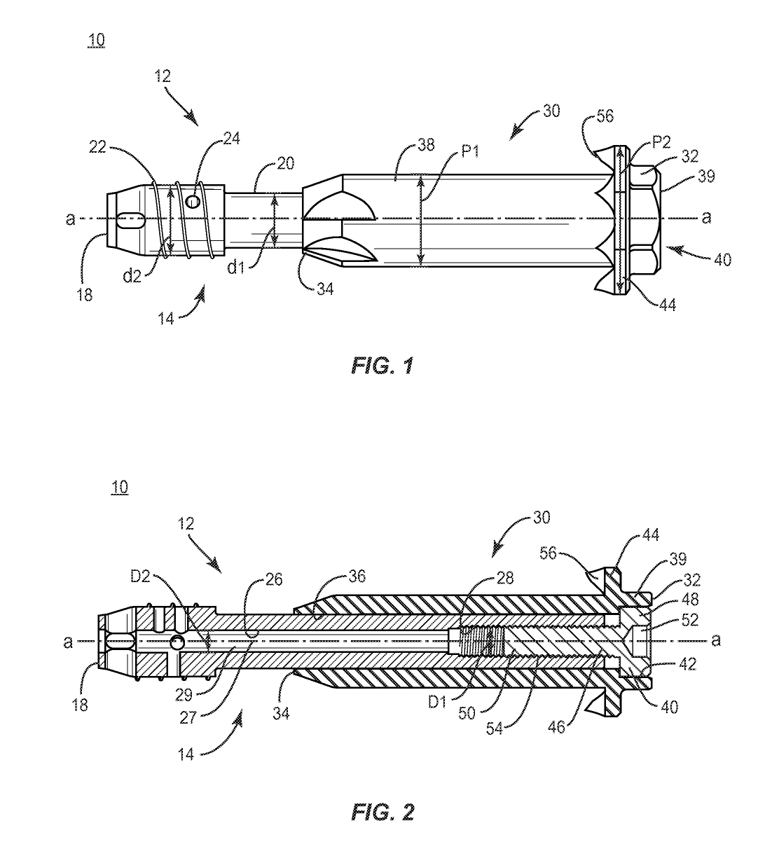

In one embodiment, a method is provided, similar to the method described below with regard to FIGS. 1-4, which comprises the steps of: making a skin mark and an incision; inserting an elongated pin through soft tissue and ilium into the sacrum under fluoroscopy; placing a cannula, with or without a handle, over the pin to protect soft tissue during subsequent drilling; drilling along the pin through the sacroiliac joint and stopping at a selected depth within the sacrum; optional tapping, for example, if screw thread is not self-tapping; optional preparation of the SI joint for accelerated fusion (for example, decorticate, preparation, clean, cause bleeding); measuring the drill depth of the bony section (for example, 50 millimeters (mm)); selecting a compression screw with a selected range of length (for example, medium screw with 45-55 mm adjustable length); setting initial screw length between 55 and 50 mm and/or at a telescoped length of 55 mm; inserting compression screw until the distal tip reaches the drill depth; shortening screw length and compressing the SI joint as the screw length reaches about 50 mm; and closing the surgical wound. In one embodiment, the final screw length after compressing the SI joint is expected between 50 and 45 mm. It is envisioned that if the screw is perforated, bone graft can be injected into the prepared joint via injection.

In one embodiment, a method is provided, similar to the method described below with regard to FIGS. 1-4, which comprises the step of pre-drilling a passage through the ilium and sacrum before inserting the screw. It is contemplated that tapping is not required as the distal screw thread can be designed for self-tapping.

In one embodiment, a method is provided, similar to the method described below with regard to FIGS. 1-4, which comprises the step of adjusting screw length prior to insertion and based on a measured depth. In one embodiment, the method can include the step of selecting a drill depth. For example, if the drill depth of the SI bone section is about 50 mm, a compression screw with an adjustable range of 55 to 45 mm can be used, which allows for measurement error and compression. It is envisioned that the telescoping length of the screw can be adjusted after implantation to achieve or enhance compression of the SI Joint. For example, the distal threaded portion of the screw is fixed in the sacrum such that shortening the telescoping length after implantation can include shortening the telescoping screw length to push the ilium toward the sacrum to cause the SI joint to narrow resulting in compression effect.

In one embodiment, the system comprises surgical navigation technology to guide drilling, tapping and screw insertion. In one embodiment, the compression screw can be cannulated for injecting biologics into the SI joint space. In one embodiment, the system comprises a nerve monitor to prevent potential nerve damage while drilling, tapping and screw insertion.

In one embodiment, the system comprises a screw that compresses the SI joint such that the gap within the joint is reduced to facilitate bone to bridge across the joint for faster fusion. It is contemplated that the SI joint surfaces are approximated to cause more resistance to their relative motions. It is further contemplated that this configuration reduces shear stresses imposed on the screw disposed across the joint, which results in more stable fixation and avoidance of screw fracture. It is further contemplated that this configuration avoids shear stress being transferred to the trans-joint screws or implants, and as such fewer and/or smaller screws can be used to stabilize the SI joint.

In one embodiment, the system comprises a screw having a washer with one or a plurality of spikes. In one embodiment, the washer has a proximal surface that includes a serration and a mating surface at the bottom of the screw head, which has serrations in an opposite direction for resistance to the screw backing out.

In one embodiment, the system comprises a screw having a washer that is retained near the bottom of the screw head via three projections. In one embodiment, the washer can include projections and threading. In one embodiment, the washer includes serration patterns on its proximal surface. In one embodiment, the washer includes serration patterns and the bottom of screw head is designed for mating and locking to resist undesirable screw back out.

It is contemplated that one or all of the components of the surgical system may be disposable, peel-pack, pre-packed sterile devices. One or all of the components of the system may be reusable. The system may be configured as a kit with multiple sized and configured components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The following discussion includes a description of a surgical system and method in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there are illustrated components of a surgical system, such as, for example, a SI implant system 10 in accordance with the principles of the present disclosure.

The components of system 10 can be fabricated from biologically acceptable materials suitable for medical applications, including metals, synthetic polymers, ceramics, bone material, tissue and/or their composites, depending on the particular application and/or preference of a medical practitioner. For example, the components of system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is configured, for example, to treat sacro-iliac joint disorders including those caused by degeneration or trauma. System 10 is adapted to immobilize opposing naturally separated surfaces of a sacro-iliac joint. System 10 includes an orthopedic implant 12. It is contemplated that implant 12 may be employed for arthrodesis applications, as will be described. In one embodiment, implant 12 has a compression screw configuration. It is envisioned that implant 12 may have alternative configurations, such as, for example, a nail, tack, post, connector or fixation element.

Implant 12 includes an inner member, such as, for example, a shaft 14. It is contemplated that shaft 14 may have a solid, hollow, porous or cage configuration. It is further contemplated that the overall and/or cross-sectional geometry of shaft 14 may have various configurations, for example, round, oval, oblong, triangular, rectangular, polygonal, irregular, uniform, non-uniform, consistent or variable. Shaft 14 defines a longitudinal axis a that extends between a first end 16 and a second end 18. End 18 is configured for penetrating a sacrum, as described herein. In one embodiment, a portion of end 18 is tapered to facilitate penetration of shaft 14 into the sacrum.

An outer surface 20 extends between ends 16 and 18. Outer surface 20 is configured for slidable engagement with a second member during axial translation, as described herein. Surface 20 is substantially even. It is contemplated that surface 20 may be variously configured, such as, for example, textured, arcuate, undulating, substantially smooth, rough, semi-porous, dimpled and/or polished.

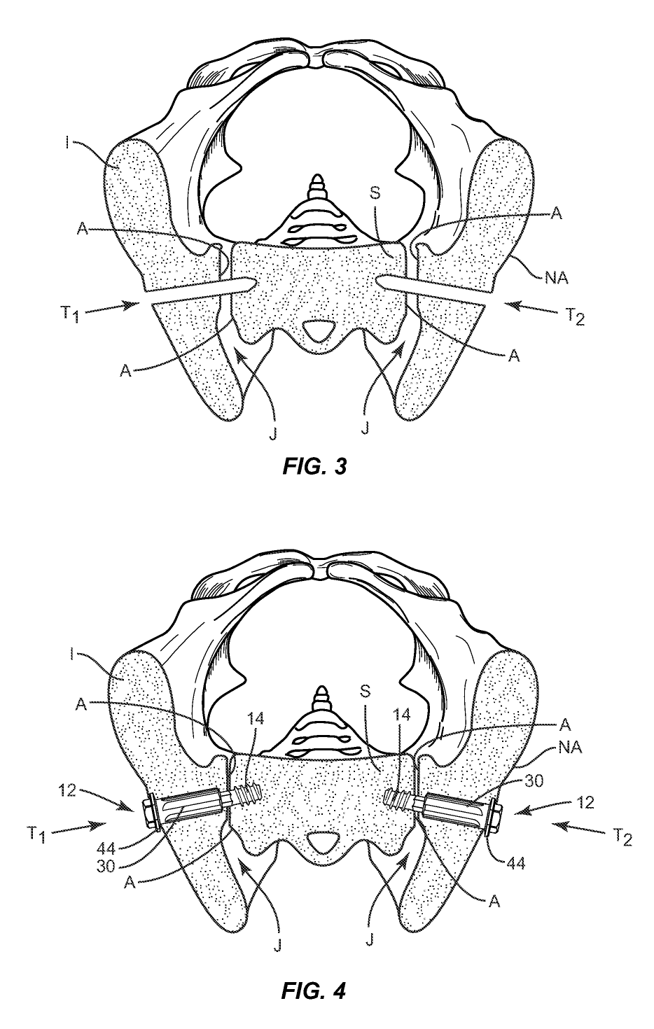

Surface 20 includes a penetrating portion, such as, for example, threaded portion 22. Portion 22 is located adjacent end 18. Portion 22 is configured for threaded fixation with a sacrum S of sacro-iliac joint J (FIGS. 3-4). Portion 22 has a cylindrical cross-section and is transarticular and penetrates tissues, including bone, of sacrum S to secure implant 12 with sacro-iliac joint J for stabilization and immobilization thereof. It is envisioned that all or only a portion of portion 22 may be disposed with sacrum S, and that a portion of portion 22 may be disposed with an ilium I of sacro-iliac joint J.

It is contemplated that portion 22 may have alternate cross-section configurations, such as, for example, those described herein. It is envisioned that all or only a portion of portion 22 may have alternate surface configurations, for alternative fixation configurations with a body cavity, such as, for example, threaded, non-threaded, arcuate, undulating, substantially smooth, rough, spiked, semi-porous, dimpled and/or polished, textured for friction fit and/or oversized for pressure fit to facilitate fixation with tissues, including bone, of sacrum S. It is further envisioned that portion 22 may include fastening elements such as anchors, barbs, spikes, detents and/or slots. In one embodiment, portion 22 includes at least one fenestration, such as for example, openings 24. Openings 24 are configured for engagement with an inner surface of shaft 14, as described herein and are configured to disperse flowable materials, such as, for example, biologics, agents, medical adhesives, bonding cements and/or bone healing substances, as described herein. It is contemplated that openings 24 may be variously configured according to the requirements of a particular application.

Shaft 14 defines a first diameter portion d1. It is contemplated that diameter d1 may be uniformly increasing or decreasing, or have alternate diameter dimensions. It is further contemplated that diameter d1 may be in a range of approximately 2 to 20 mm, and preferably in a range of approximately 5 to 15 mm. Shaft 14 includes a second greater diameter portion d2 located adjacent end 18. It is contemplated that diameter d2 may be uniformly increasing or decreasing, or have alternate diameter dimensions. It is further contemplated that diameter d2 may be in a range of approximately 2 to 25 mm, and preferably in a range of approximately 5 to 20 mm.

Shaft 14 includes an inner surface 26. Inner surface 26 defines a passageway, such as, for example, channel 27, such that implant 12 is cannulated. Channel 27 extends between end 16 and 18. Channel 27 includes threads 28 adjacent end 16. Threads 28 are configured for engagement with an actuator, as described herein. Channel 27 includes a substantially even portion 29 adjacent end 18. Channel 27 has a first diameter D1 that is adjacent end 16 and a second smaller diameter D2 adjacent end 18. It is contemplated that diameters D1 and D2 may be uniformly increasing or decreasing, or have alternate diameter dimensions. Channel 27 is configured to disperse flowable materials, such as, for example, biologics, agents, medical adhesives, bonding cements and/or bone healing substances, as described herein.

Implant 12 includes an outer member, such as, for example, sleeve 30. Sleeve 30 is configured for slidable engagement with shaft 14 during axial translation and is configured for disposal within a body cavity formed in sacro-iliac joint J. It is contemplated that sleeve 30 may have a solid, hollow, porous or cage configuration. It is further contemplated that the overall and/or cross-sectional geometry of sleeve 30 may have various configurations, for example, round, oval, oblong, triangular, rectangular, polygonal, irregular, uniform, non-uniform, consistent or variable. Sleeve 30 is positioned along longitudinal axis a and extends between a first end 32 and a second end 34. End 32 is configured for engagement with an outer non-articular surface of an ilium (FIGS. 3-4), as described herein. End 34 is tapered to facilitate penetration of shaft 14 into sacrum S. In one embodiment, end 34 is non-tapered and uniform in diameter. End 34 includes axially oriented ribs 35 disposed circumferentially about an outer surface 38 of shaft 14. It is envisioned that end 34 may include one or a plurality of ribs. In one embodiment, end 34 has an even outer surface.

Sleeve 30 includes an inner surface 36. Surface 36 is substantially even such that shaft 14 slideably engages sleeve 30 during axial translation. It is contemplated that surface 36 may be variously configured, such as, for example, textured, arcuate, undulating, substantially smooth, rough, semi-porous, dimpled and/or polished.

Outer surface 38 defines a first diameter portion P1 and a second greater diameter portion P2. Diameter P2 is adjacent end 32. It is contemplated that diameters D1 and D2 may be uniformly increasing or decreasing, or have alternate diameter dimensions.

End 32 includes an axial flange 39. Flange 39 defines a cavity 40 configured for disposal of an actuator, as described herein. Cavity 40 includes an inner surface 42. It is contemplated that all or only a portion of surface 42 may have alternate surface configurations for engagement with the actuator, such as, for example, those alternatives described herein.

Sleeve 30 includes a transverse flange 44, which is configured for engagement with non-articular surface NA to facilitate compression of SI joint J, as will be described. Flange 44 includes at least one fixation element, such as, for example, spikes 56. Spikes 56 are configured to penetrate the outer surface of ilium I. It is envisioned that flange 44 may have alternate gripping configurations, such as, for example, anchors, barbs, detents, openings, arcuate, undulating, rough, serrations, semi-porous, dimpled and/or textured to facilitate fixation with tissues, including bone.

Implant 12 includes an actuator, such as, for example, screw 46. Screw 46 is configured for rotational movable engagement with shaft 14 and sleeve 30 to cause axial translation of shaft 14 relative to sleeve 30 such that separated articular surfaces of sacrum S and ilium I are drawn into fixation. Screw 46 includes a first end, such as, for example, a head 48 and a second end, such as, for example, a shaft 50. Head 48 defines a recess, such as, for example, a tool socket 52. Socket 52 is configured for engagement with a medical tool, such as, for example, a driver (not shown), which engages implant 12 so that implant 12 can be driven into an implant space prepared by a medical device. It is contemplated that socket 52 may be variously shaped, such as, for example, hexagonal, star, square, slotted, indented hexagon, cross and/or slotted and squared. Shaft 50 defines a threaded outer surface 54. Surface 54 is configured for movable male/female engagement with threads 28 of channel 27.

As sleeve 30 is relatively rotated about shaft 14, threaded outer surface 54 and threads 28 cooperatively engage such that sleeve 30 moves relative to shaft 14 along longitudinal axis a to facilitate axial translation of sleeve 30. With end 18 anchored in sacrum S, such axial translation of sleeve 30 causes spikes 56 of flange 44 to be drawn into engagement with outer non-articular surface NA of ilium I. With end 18 anchored in sacrum S and surface 56 disposed in engagement with outer non-articular surface NA, further rotation of sleeve 30 relative to shaft 14, axially translates sleeve 30 relative to shaft 14 in a configuration to draw separated articular surfaces A of sacrum S and ilium I into fixation to immobilize sacro-iliac joint J. In one embodiment, implant 12 is disposable between a first orientation, such as, for example, a non-compression orientation, as shown in FIG. 3, such that articular surfaces A of SI joint J are spaced apart and a second orientation, such as, for example, a compression orientation, as shown in FIG. 4, such that articular surfaces A are drawn into engagement.

It is envisioned that shaft 18, sleeve 30 and/or screw 46 can be variously configured and dimensioned with regard to size, shape, thickness, geometry and material. Each of shaft 18, sleeve 30 and/or screw 46 may also be formed of one or a plurality of elements such as spaced apart portions, staggered patterns and mesh. It is envisioned that the particular geometry and material parameters of shaft 14, sleeve 30 and/or screw 46 may be selected to modulate the flexibility or stiffness of implant 12, such as those examples discussed herein. For example, each of shaft 14, sleeve 30 and/or screw 46 can be configured to have varying ranges or degrees of flexibility or stiffness such as rigid, compliant, or reinforced. Depending on the flexibility or stiffness of shaft 14, sleeve 30 and/or screw 46, the flexibility or stiffness of implant 12 can be contoured according to the requirements of a particular application. It is contemplated that the ability to vary stiffness of implant 12 promotes fusion of the elements of sacro-iliac joint J. It is envisioned that the components of implant 12 may be monolithically formed, integrally connected or arranged with attaching elements.

It is contemplated that the system 10 can include a plurality of sacro-iliac implants 12, similar to that described above with regard to FIGS. 1-2. In one embodiment, system 10 can include a pair of sacro-iliac implants 12. In one embodiment, system 10 includes three sacro-iliac implants 12. It is contemplated that employing the plurality of sacro-iliac implants 12 can optimize the stability of sacro-iliac joint J. The plurality of sacro-iliac implants 12 can be inserted through the same or an alternate trajectory. The plurality of sacro-iliac implants 12 can be oriented in a side by side engagement, spaced apart and/or staggered. It is envisioned that one or all of the plurality of sacro-iliac implants 12 may be inserted via a trajectory oriented from an anterior, posterior, superior and/or inferior direction, similar to that described herein. It is further envisioned that one or a plurality of sacro-iliac implants 12 may be used.

In assembly, operation and use, system 10 including sacro-iliac implant 12, described herein, is employed with a surgical procedure for treatment of a sacro-iliac joint J of a patient. System 10 may also be employed with other surgical procedures, which may include one or all of the steps described herein. In particular, system 10 is employed with a surgical arthrodesis procedure, such as, for example, fusion for treatment of an applicable condition or injury of an affected sacro-iliac joint J, as shown in FIGS. 3-4. It is contemplated that system 10 including sacro-iliac implant 12 may be employed during a surgical fusion procedure for treatment of a condition or injury, such as, degeneration or fracture.

In use, to treat the affected section of sacro-iliac joint J, a medical practitioner obtains access to a surgical site including sacro-iliac joint J in any appropriate manner, such as through incision and retraction of tissues. It is envisioned that system 10 may be used in any existing surgical method or technique including open surgery, mini-open surgery, minimally invasive surgery and percutaneous surgical implantation, whereby sacro-iliac joint J is accessed through a mini-incision, or sleeve that provides a protected passageway to the area. Once access to the surgical site is obtained, the particular surgical procedure is performed for treating the sacro-iliac joint disorder. System 10 is then employed to augment the surgical treatment. System 10 can be delivered or implanted as a pre-assembled device or can be assembled in situ. System 10 may be completely or partially revised, removed or replaced in situ. It is contemplated that one or all of the components of system 10 can be delivered to the surgical site via manual manipulation and/or a free hand technique.

Trajectories T₁, T₂, as shown in FIG. 3, are defined for insertion of the components of sacro-iliac implant 12 within sacro-iliac joint J. It is envisioned that trajectory T₁ may be oriented perpendicular, parallel, angularly offset, offset, cruciate and/or staggered relative to trajectory T₂. The components of sacro-iliac implant 12 are inserted via the protected passageway along each of the defined trajectories T₁, T₂ into sacro-iliac joint J. Separate body cavities of sacro-iliac joint J are prepared along each of trajectories T₁, T₂ for disposal of sacro-iliac implant 12. It is contemplated that a guide wire and/or a trocar-cannula assembly may be employed as an instrument for gaining access to the surgical site and/or defining the trajectories.

The protected passageway includes a dilator/delivery tube (not shown) configured to deliver sacro-iliac implant 12 adjacent to the joint space of sacro-iliac joint J. It is envisioned that the dilator/delivery tube may be configured as an in-situ guidable instrument, and may include an endoscope camera tip for viewing insertion trajectory. It is further envisioned that the components of sacro-iliac implant 12 may include a cavity configured to receive the instrument to facilitate delivery of sacro-iliac implant 12 to sacro-iliac joint J. It is contemplated that the components of sacro-iliac implant 12 and/or other components of system 10, and the several embodiments of system 10 described herein, may include a connecting portion, opening and/or mechanism, such as, for example, threaded holes, snap-on connects, and quick-connect mechanisms for connection to a delivery instrument for implant disposal, detachable connection and release and removal from the surgical site.

The body cavities are tapped and/or drilled in the joint surfaces of sacro-iliac joint J in an orientation and alignment with sacrum S and ilium I. It is contemplated that a guide instrument (not shown) may be used to facilitate formation of such cavities by providing an alignment device for a surgical drill and/or tap. A first sacro-iliac implant 12 including shaft 14, with or without sleeve 30, is delivered via the guide instrument to sacro-iliac joint J into alignment with the body cavity along trajectory T₁, as shown in FIG. 4. A second sacro-iliac implant 12 is similarly delivered via the guide instrument to sacro-iliac joint J into alignment with the body cavity along trajectory T₂. For each sacro-iliac implant 12, threaded portion 22 is threaded with the joint surfaces of sacro-iliac joint J such that outer surface 20 is fixed with the tissues of sacrum S, as described above.

Sleeve 30 is mounted with shaft 14 and screw 46 such that outer surface 20 is slidably supported by inner surface 36. Threaded outer surface 54 of screw 46 is rotatably received by threads 28 of channel 27 in a male/female engagement. Sleeve 30 is manipulated via socket 52 by a practitioner and/or an appropriate instrument, such as, for example, a driver, to relatively rotate about shaft 14 such that sleeve 30 moves relative to shaft 14 along longitudinal axis a to facilitate axial translation of sleeve 30, as described above.

With end 18 anchored in sacrum S via threaded portion 22, axial translation of sleeve 30 causes flange 44 to be drawn into engagement with outer non-articular surface NA of ilium I. With end 18 anchored in sacrum S and spikes 56 disposed in engagement with outer non-articular surface NA, further rotation of sleeve 30 relative to shaft 14, axially translates sleeve 30 relative to shaft 14 in a configuration to draw separated articular surfaces A of sacrum S and ilium I into fixation to secure, stabilize and immobilize sacro-iliac joint J, as shown in FIG. 4, for arthrodesis. Upon completion of the procedure, the non-implant components of system 10 are removed from the surgical site and the incision is closed.

In one embodiment, system 10 includes fastening elements, such as, for example, screws (not shown) configured for fixation with articular surfaces A external to sacro-iliac implant 12. The screws are employed to secure joint surfaces and provide complementary stabilization and immobilization to sacro-iliac joint J. It is envisioned that sacro-iliac implant 12 may include locking structure such as, for example, clips, hooks, adhesives and/or flanges. It is further envisioned that in joint fusion applications, the components of sacro-iliac implant 12 includes voids, cavities and/or openings for including therapeutic polynucleotides or polypeptides and bone growth promoting material, such as those described herein, which can be packed or otherwise disposed therein.

For example, the components of sacro-iliac implant 12 may include at least one agent including biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, biologically active agents coated onto the exterior of the components of sacro-iliac implant 12 and/or applied thereto for gradual release such as by blending in a bioresorbable polymer that releases the biologically active agent or agents in an appropriate time dependent fashion as the polymer degrades within the patient. Suitable biologically active agents include, for example, bone morphogenic protein (BMP), cytokines, osteoconductive and/or osteoinductive material such as HA and/or osteoinductive agent, either partially or completely to enhance osteointegration and fusion across sacro-iliac joint J, and/or for enhanced bony fixation to the treated area. It is contemplated that such materials may include a combination of materials such as, for example, an HA coating with BMP for improved fusion rate. It is further contemplated that such materials may include pharmacological agents as described herein. It is envisioned that the components of the sacro-iliac implant system may include large cavities or slots configured to receive fasteners and/or pack bone graft, such as, for example, autograft, allograft, bone chips, demineralized bone matrix, calcium phosphate, HA, and bone growth agents in a carrier matrix for enhancing fusion.

The components of sacro-iliac implant 12 can be made of radiolucent materials such as polymers. Radiomarkers may be included for identification under x-ray, fluoroscopy, CT or other imaging techniques.

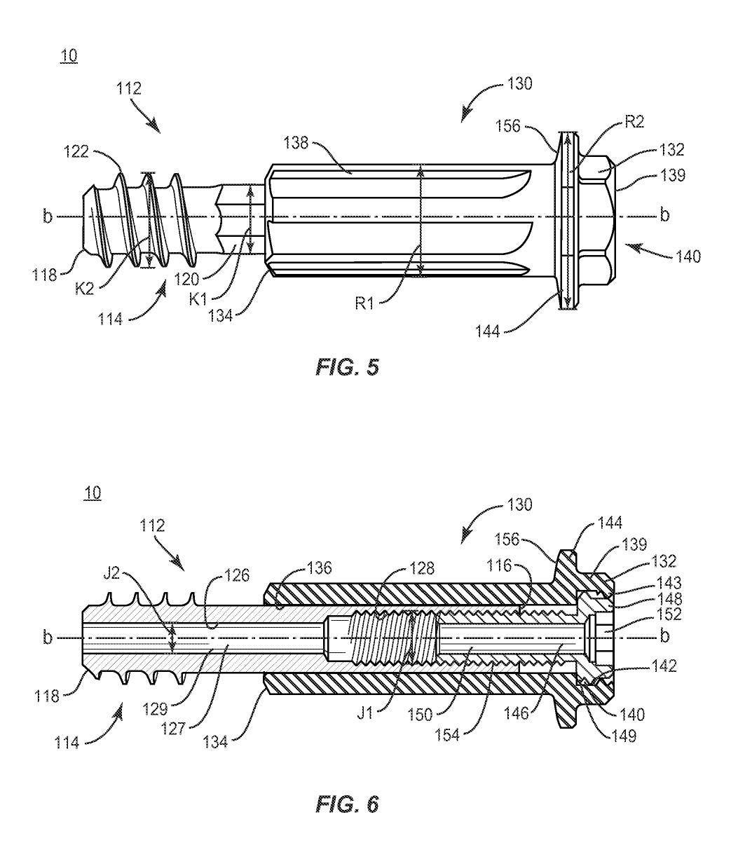

In one embodiment, as shown in FIGS. 5-6, system 10 includes an implant 112, similar to the systems and methods described above, which includes an inner member, such as, for example, shaft 114. It is contemplated that shaft 114 may have a solid, hollow, porous or cage configuration. It is further contemplated that the overall and/or cross-sectional geometry of shaft 114 may have various configurations, for example, round, oval, oblong, triangular, rectangular, polygonal, irregular, uniform, non-uniform, consistent or variable. Shaft 114 defines a longitudinal axis b that extends between a first end 116 and a second end 118. End 118 is configured for penetrating a sacrum, as described herein.

An outer surface 120 extends between ends 116 and 118. Outer surface 120 is configured for slidable engagement with a second member during axial translation, as described herein. Surface 120 is substantially even.

Surface 120 includes a penetrating portion, such as, for example, threaded portion 122. Portion 122 is located adjacent end 118. Portion 122 is configured for threaded fixation with sacrum S of sacro-iliac joint J (FIGS. 3-4). Portion 122 has a cylindrical cross-section and is transarticular and penetrates tissues, including bone, of sacrum S to secure implant 112 with sacro-iliac joint J for stabilization and immobilization thereof. It is envisioned that all or only a portion of portion 122 may be disposed with sacrum S, and that a portion of portion 122 may be disposed with ilium I of sacro-iliac joint J.

It is contemplated that portion 122 may have alternate cross-section configurations, such as, for example, those described herein. It is envisioned that all or only a portion of portion 122 may have alternate surface configurations, for alternative fixation configurations with a body cavity, such as, for example, non-threaded, arcuate, undulating, substantially smooth, rough, spiked, semi-porous, dimpled and/or polished, textured for friction fit and/or oversized for pressure fit to facilitate fixation with tissues, including bone, of sacrum S. It is further envisioned that portion 122 may include fastening elements such as anchors, barbs, spikes, detents, slots and/or openings.

Shaft 114 defines a first diameter portion k1. It is contemplated that diameter k1 may be uniformly increasing or decreasing, or have alternate diameter dimensions. It is further contemplated that diameter k1 may be in a range of approximately 2 to 25 mm, and preferably in a range of approximately 5 to 20 mm. Shaft 114 includes a second greater diameter portion k2 located adjacent end 118. It is contemplated that diameter k2 may be uniformly increasing or decreasing, or have alternate diameter dimensions. It is further contemplated that diameter k2 may be in a range of approximately 2 to 30 mm, and preferably in a range of approximately 5 to 25 mm.

Shaft 114 includes an inner surface 126. Inner surface 126 defines a passageway, such as, for example, channel 127. Channel 127 extends between end 116 and 118. Channel 127 includes threads 128 adjacent end 116. Threads 128 are configured for engagement with an actuator as described herein. Channel 127 includes a substantially even portion 129 adjacent end 118. Channel 127 has a first diameter j1 that is adjacent end 116 and a second smaller diameter j2 adjacent end 118. It is contemplated that diameters j1 and j2 may be uniformly increasing or decreasing, or have alternate diameter dimensions. Channel 127 is configured to disperse flowable materials, such as, for example, biologics, agents, medical adhesives, bonding cements and/or bone healing substances, as described herein.

Implant 112 includes an outer member, such as, for example, sleeve 130. Sleeve 130 is configured for slidable engagement with shaft 114 during axial translation and is configured for disposal within a body cavity formed in sacro-iliac joint J. It is contemplated that sleeve 130 may have a solid, hollow, porous or cage configuration. It is further contemplated that the overall and/or cross-sectional geometry of sleeve 130 may have various configurations, for example, round, oval, oblong, triangular, rectangular, polygonal, irregular, uniform, non-uniform, consistent or variable. Sleeve 130 extends between a first end 132 and a second end 134. End 132 is configured for engagement with an outer non-articular surface of an ilium, as described herein. Sleeve 130 is positioned along longitudinal axis b.

Sleeve 130 includes an inner surface 136. Surface 136 is substantially even such that shaft 114 slideably engages sleeve 130 during axial translation. It is contemplated that surface 136 may be variously configured, such as, for example, textured, arcuate, undulating, substantially smooth, rough, semi-porous, dimpled and/or polished.

An outer surface 138 defines a first diameter portion R1 and a second greater diameter portion R2. Diameter R2 is adjacent end 132. It is contemplated that diameters R1 and R2 may be uniformly increasing or decreasing, or have alternate diameter dimensions.

End 132 includes an axial flange 139. Flange 139 defines a cavity 140 configured for disposal of an actuator, as described herein. Cavity 140 includes an inner surface 142. Surface 142 includes a thread form 143 for engagement with the actuator to facilitate disposal of the actuator with cavity 140, as will be described. It is contemplated that all or only a portion of surface 142 may have alternate surface configurations for engagement with the actuator, such as, for example, those alternatives described herein.

Sleeve 130 includes a transverse flange 144. Flange 144 includes a substantially even outer surface 156 and is configured to fix with the outer surface of the Ilium I. It is envisioned that flange 144 may have alternate gripping configurations, such as, for example, spikes, anchors, barbs, detents, openings, arcuate, undulating, rough, serrations, semi-porous, dimpled and/or textured to facilitate fixation with tissues, including bone.

Implant 112 includes an actuator, such as, for example, screw 146. Screw 146 is configured for rotational movable engagement with shaft 114 and sleeve 130 to cause axial translation of shaft 114 relative to sleeve 130 such that separated articular surfaces of sacrum S and ilium I are drawn into fixation. Screw 146 includes a first end, such as, for example, head 148 and a second end, such as, for example, shaft 150. Head 148 defines a recess, such as, for example, tool socket 152. Socket 152 is configured for engagement with a medical tool, such as, for example, a driver (not shown), which attaches to implant 112 so that implant 112 can be driven into an implant space prepared by a medical device. It is contemplated that socket 152 may be variously shaped, such as, for example, hexagonal, star, square, slotted, indented hexagon, cross and/or slotted and squared.

Head 148 includes an outer surface defining a thread form 149 configured for engagement with thread form 143. Head 148 is rotatably threaded with surface 142 via engagement of threads 143, 149 for axial translation of head 148 and disposal within cavity 140. Upon disposal of head 148 within cavity 140 such that the distal outer surface of head 148 is in a flush engagement with surface 142, head 148 is freely rotatable relative to surface 142 to facilitate axial translation of shaft 114 relative to sleeve 130 and compression of articular surfaces A.

Shaft 150 defines a threaded outer surface 154. Surface 154 is configured for movable male/female engagement with threads 128 of channel 127. As sleeve 130 is relatively rotated about shaft 114, threaded outer surface 154 and threads 128 cooperatively engage such that sleeve 130 moves relative to shaft 114 along longitudinal axis b to facilitate axial translation of sleeve 130. With end 118 anchored in sacrum S, axial translation of sleeve 130 causes surface 156 of flange 144 to be drawn into engagement with outer non-articular surface NA of ilium I. With end 118 anchored in sacrum S and surface 156 disposed in engagement with outer non-articular surface NA, further rotation of sleeve 130 relative to shaft 114, axially translates sleeve 130 relative to shaft 114 in a configuration to draw separated articular surfaces A of sacrum S and ilium I into fixation to immobilize sacro-iliac joint J, similar to the non-compression and compression orientations of implant 12 described above.

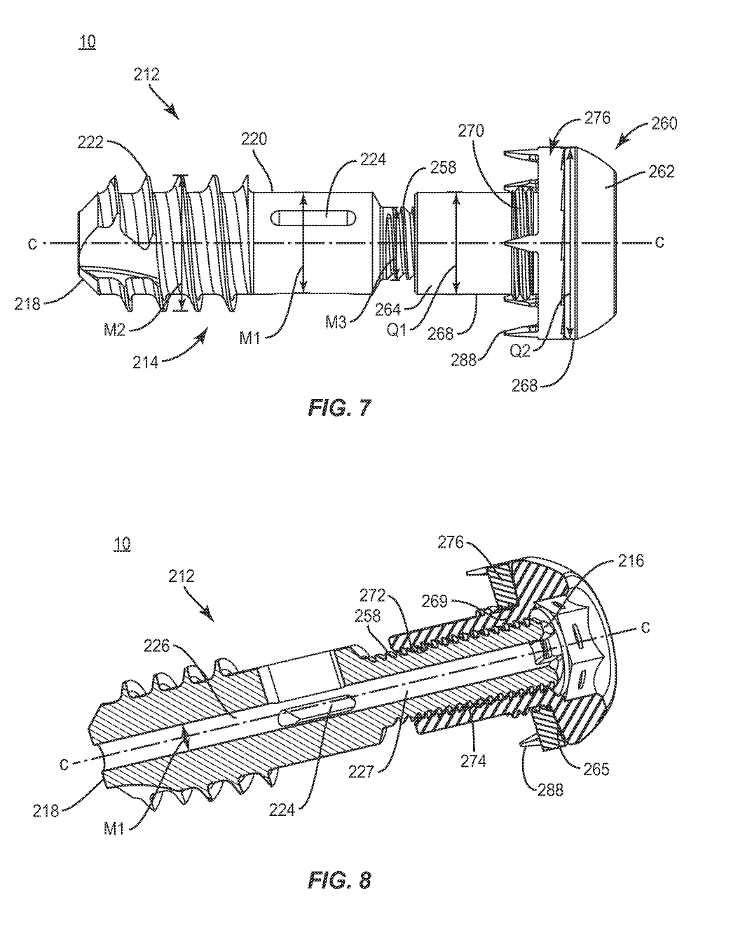

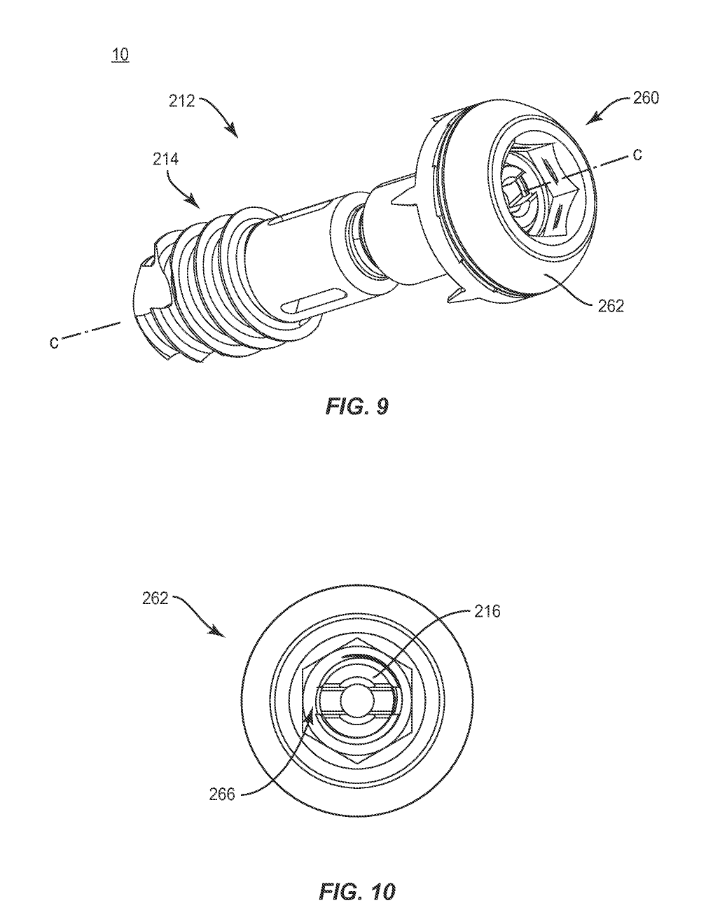

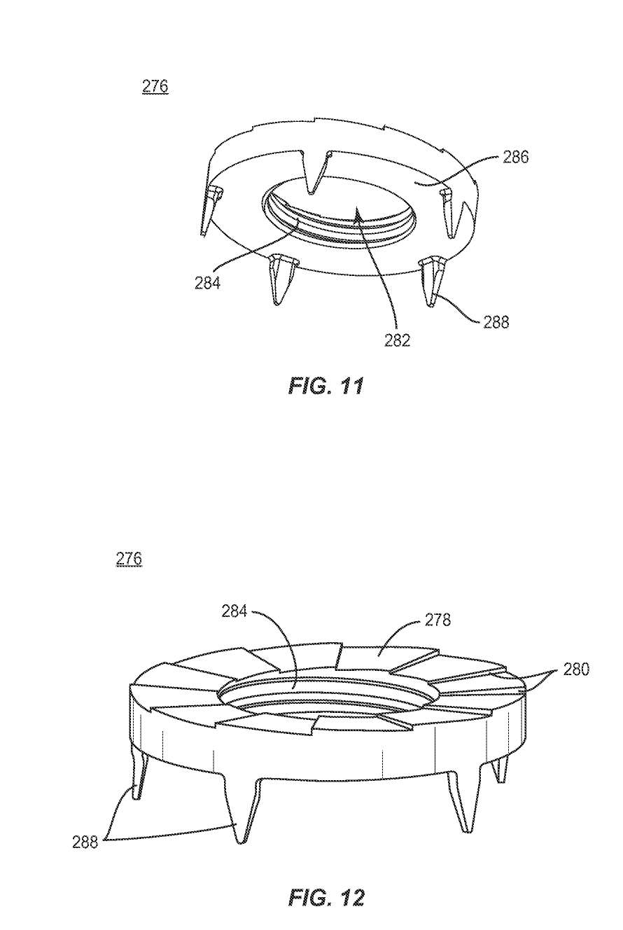

In one embodiment, as shown in FIGS. 7-12, system 10 includes an implant 212, similar to the systems and methods described above, which includes an inner member, such as, for example, shaft 214. It is contemplated that shaft 214 may have a solid, hollow, porous or cage configuration. It is further contemplated that the overall and/or cross-sectional geometry of shaft 214 may have various configurations, for example, round, oval, oblong, triangular, rectangular, polygonal, irregular, uniform, non-uniform, consistent or variable. Shaft 214 defines a longitudinal axis c that extends between a first end 216 and a second end 218 of member 214. End 218 is configured for penetrating a sacrum, as described herein.

An outer surface 220 extends between ends 216 and 218. At least a portion of outer surface 220 is configured for threaded engagement with a second member during axial translation, as described herein. It is contemplated that surface 220 may be variously configured, such as, for example, textured, arcuate, undulating, substantially smooth, rough, semi-porous, dimpled and/or polished.

Surface 220 includes a penetrating portion, such as, for example, first threaded portion 222. Portion 222 is located adjacent end 218. Portion 222 is configured for threaded fixation with sacrum S of sacro-iliac joint J (FIGS. 3-4). Portion 222 has a cylindrical cross-section and is transarticular and penetrates tissues, including bone, of sacrum S to secure implant 212 with sacro-iliac joint J for stabilization and immobilization thereof. It is envisioned that all or only a portion of portion 222 may be disposed with sacrum S, and that a portion of portion 222 may be disposed with ilium I of sacro-iliac joint J.

It is contemplated that portion 222 may have alternate cross-section configurations, such as, for example, those described herein. It is envisioned that all or only a portion of portion 222 may have alternate surface configurations, for alternative fixation configurations with a body cavity, such as, for example, threaded, non-threaded, arcuate, undulating, substantially smooth, rough, spiked, semi-porous, dimpled and/or polished, textured for friction fit and/or oversized for pressure fit to facilitate fixation with tissues, including bone, of sacrum S. It is further envisioned that portion 222 may include fastening elements such as anchors, barbs, spikes, detents and/or slots. In one embodiment, portion 222 includes at least one fenestration, such as for example, openings 224.

Surface 220 includes a second threaded portion 258. Portion 258 is located adjacent end 216. Portion 258 is configured for threaded engagement during axial translation with a second member, as described herein.

Shaft 214 defines a first diameter portion m1. It is contemplated that diameter m1 may be uniformly increasing or decreasing, or have alternate diameter dimensions. It is further contemplated that diameter m1 may be in a range of approximately 2 to 20 mm, and preferably in a range of approximately 5 to 15 mm. Shaft 214 includes a second greater diameter portion m2 located adjacent end 218. It is contemplated that diameter m2 may be uniformly increasing or decreasing, or have alternate diameter dimensions. It is further contemplated that diameter m2 may be in a range of approximately 2 to 25 mm, and preferably in a range of approximately 5 to 20 mm. Shaft 214 includes a third minor diameter m3. It is contemplated that diameter m3 may be uniformly increasing or decreasing, or have alternate diameter dimensions. It is further contemplated that diameter m3 may be in a range of approximately 1 to 15 mm, and preferably in a range of approximately 3 to 10 mm.

Shaft 214 includes an inner surface 226, which is substantially even. Inner surface 226 defines a passageway, such as, for example, channel 227. Channel 227 extends between end 216 and 218. Channel 227 has diameter m1 throughout. It is contemplated that diameter m1 may be uniformly increasing or decreasing, or have alternate diameter dimensions. Channel 227 is configured to disperse flowable materials, such as, for example, biologics, agents, medical adhesives, bonding cements and/or bone healing substances, as described herein.

Implant 212 includes an actuator, such as, for example, screw 260. Screw 260 is configured for rotational threaded engagement with shaft 214 to cause axial translation of shaft 214 relative to screw 260 such that separated articular surfaces of the sacrum and the ilium are drawn into fixation and is configured for disposal within a body cavity formed in sacro-iliac joint J.

Screw 260 includes a first end, such as, for example, head 262 and a second end, such as, for example, shaft 264. A surface 265 of head 262 is serrated and configured for engagement with a fixation element, as described herein. Head 262 defines a recess, such as, for example, tool socket 266. Socket 266 is configured for engagement with a medical tool, such as, for example, a driver (not shown), which attaches to implant 212 so that implant 212 can be driven into an implant space prepared by a medical device. It is contemplated that socket 266 may be variously shaped, such as, for example, hexagonal, star, square, slotted, indented hexagon, cross and/or slotted and squared.

Shaft 264 and head 262 define an outer surface 268. Surface 268 is substantially even. Surface 268 includes a threaded portion 270 adjacent head 262. Portion 270 is configured for engagement with a fixation element, as described herein. It is contemplated that surface 268 may be variously configured, such as, for example, textured, arcuate, undulating, substantially smooth, rough, semi-porous, dimpled and/or polished.

Outer surface 268 defines a first diameter portion Q1 and a second greater diameter portion Q2. Diameter Q2 is adjacent head 262. It is contemplated that diameters Q1 and Q2 may be uniformly increasing or decreasing, or have alternate diameter dimensions.

Shaft 264 includes an inner surface 272. Surface 272 includes threads 274. Threads 274 are configured for engagement with threaded portion 258 of shaft 214 to facilitate axial translation of shaft 214 relative to screw 260.

A fixation element, such as, for example, a washer 276 is configured for threaded engagement with screw 260 and to penetrate the outer surface of Ilium I. Washer 276 defines a first outer surface 278. Surface 278 includes serrations 280. Serrations 280 are configured for mated engagement with serrated surface 265 of head 262. In one embodiment, the engagement of the serrated surfaces prevents backout of the components of implant 212.

Washer 276 includes an opening 282. Opening 282 includes a threaded portion 284. Portion 284 is configured for threaded engagement with threaded portion 270 of shaft 264. A second outer surface 286 defines at least one penetration element, such as, for example, spikes 288, similar to the spikes described above with regard to FIGS. 1-4. Spikes 288 are configured for penetrating the outer surface of Ilium I. It is envisioned that surface 286 may have alternate gripping configurations, such as, for example, hooks, anchors, barbs, detents, openings, arcuate, undulating, rough, serrations, semi-porous, dimpled and/or textured to facilitate fixation with tissues, including bone.

Threads 284 are configured for engagement with threads 270. Washer 276 is rotatably threaded with surface 268 via engagement of threads 270, 284 for axial translation of washer 276 and disposal along an even surface 269 of surface 268. Upon disposal of washer 276 with surface 269 such that the proximal outer surface of washer 276 is in a contacting engagement with surface 265, washer 276 is freely rotatable relative to surface 269 to facilitate penetration of spikes 288 with the outer surface of Ilium I. Upon penetration of spikes 288 with ilium I, the serrated surfaces of washer 276 and screw 260 are drawn into a fixed engagement. In one embodiment, implant 212 does not include washer 276 and surface 265 directly engages the outer surface of ilium I. It is envisioned that surface 265 may be substantially even.

As screw 260 is relatively rotated about shaft 214, threads 274 and threads 258 cooperatively engage such that shaft 214 moves relative to screw 260 along longitudinal axis c to facilitate axial translation of shaft 214. With end 218 anchored in sacrum S, axial translation of screw 260 causes washer 276 to be drawn into fixation with outer non-articular surface NA of ilium I, as described. With end 218 anchored in sacrum S and washer 276 disposed in engagement with outer non-articular surface NA, further rotation of screw 260 relative to shaft 214, axially translates shaft 214 relative to screw 260 in a configuration to draw separated articular surfaces A of sacrum S and ilium I into fixation to immobilize sacro-iliac joint J, similar to the non-compression and compression orientations described above.

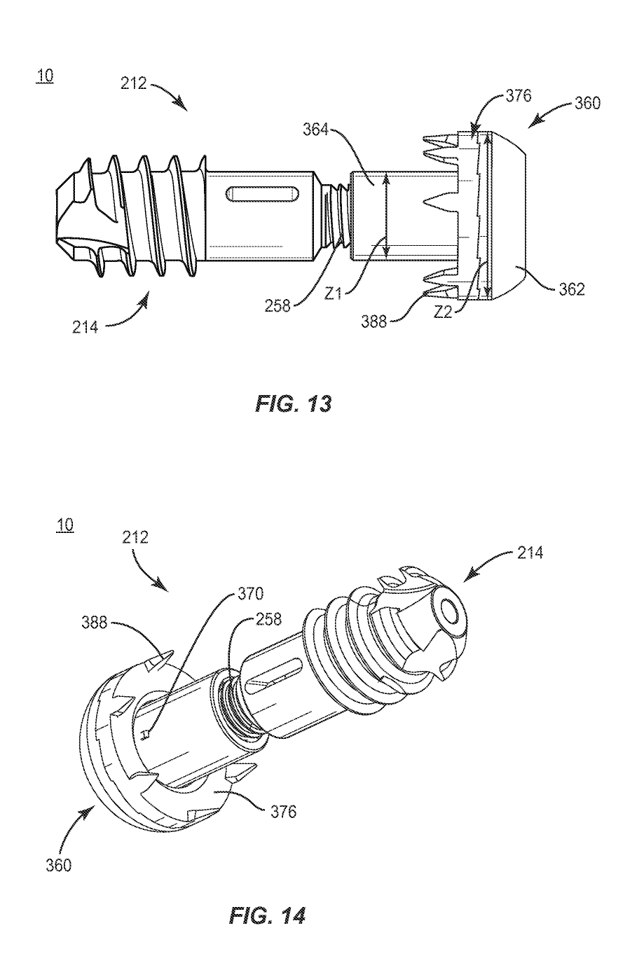

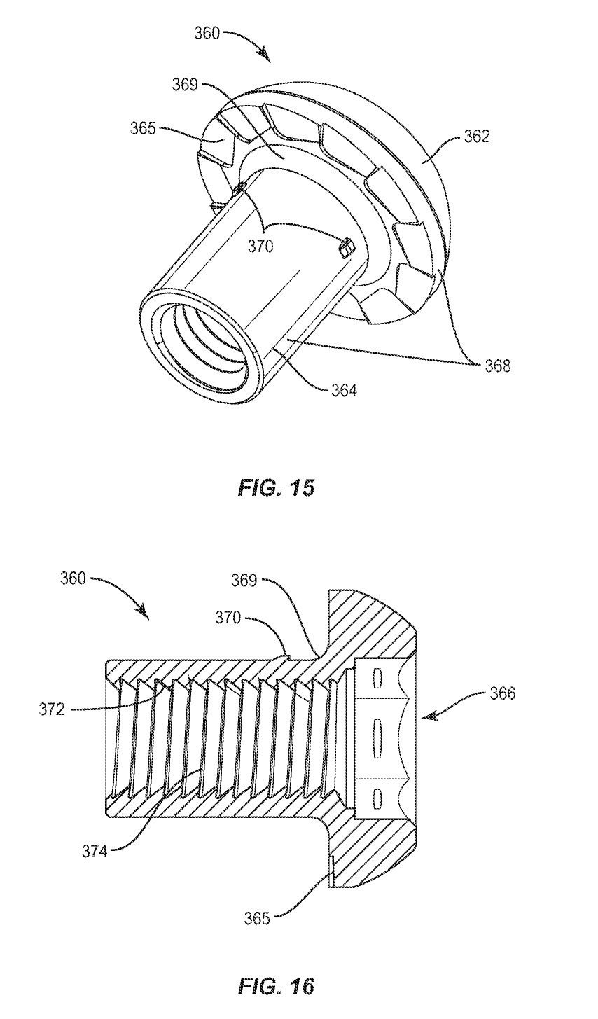

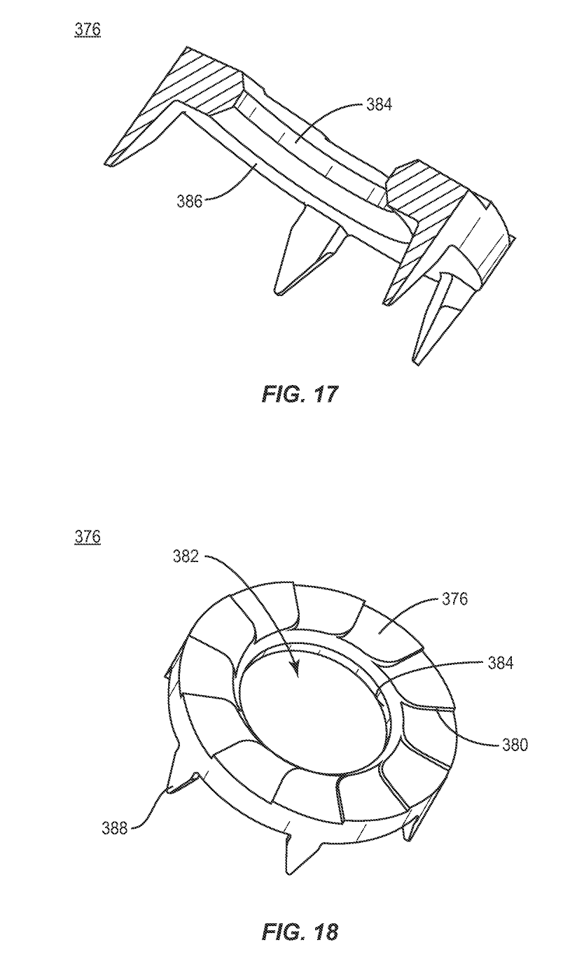

In one embodiment, as shown in FIGS. 13-18, implant 212 described above with regard to FIGS. 7-12, includes an actuator, such as, for example, a screw 360. Screw 360, similar to screw 260, is configured for rotational threaded engagement with shaft 214 to cause axial translation of shaft 214 relative to screw 360 such that separated articular surfaces of the sacrum and the ilium are drawn into fixation and is configured for disposal within a body cavity formed in sacro-iliac joint J, similar to the systems and methods described above.

Screw 360 includes a head 362 and a shaft 364. A surface 365 of head 362 is serrated and configured for engagement with a washer 376, described below. Head 362 defines a tool socket 366. Socket 366 is configured for engagement with a medical driver (not shown), which attaches to implant 212 so that implant 212 can be driven into an implant space prepared by a medical device.

Shaft 364 and head 362 define an outer surface 368. Surface 368 includes engagement portions, such as, for example, protrusions 370 adjacent head 362. Protrusions 370 are configured for engagement with washer 376.

Shaft 364 includes an inner surface 372. Surface 372 includes threads 374. Threads 374 are configured for engagement with threaded portion 258 of shaft 214 to facilitate axial translation of shaft 214 relative to screw 360.

Washer 376 is configured for threaded engagement with screw 360 and to penetrate the outer surface of Ilium I. Washer 376 defines a first outer surface 378. Surface 378 includes serrations 380. Serrations 380 are configured for mated engagement with serrated surface 365 of head 362.

Washer 376 includes an opening 382. Opening 382 includes a circumferential lip 384. Lip 384 is configured for a locking engagement with protrusions 370 of washer 376, as will be described. A second outer surface 386 defines at least one penetration element, such as, for example, spikes 388. Spikes 388 are configured for penetrating the outer surface of Ilium I. It is envisioned that lip 384 may extend about all or only a portion of the entire circumference of opening 382.

Washer 376 is forced over protrusions 370 for disposal of washer 376 along a surface 369 of surface 368. In one embodiment, washer 376 is forced over protrusions 370 in a snap fit engagement. In one embodiment, the flexibility of lip 384 and/or protrusions 370 facilitates deformation of lip 384 and/or protrusions 370 such that washer 376 slides over protrusions 370 and into position with surface 369.

Upon disposal of washer 376 with surface 369 such that the proximal outer surface of washer 376 is in a contacting engagement with surface 365, washer 376 is freely rotatable relative to surface 369 to facilitate penetration of spikes 388 with the outer surface of Ilium I. Protrusions 370 lock washer 376 with screw 360. Upon penetration of spikes 388 with ilium I, the serrated surfaces of washer 376 and screw 360 are drawn into a fixed engagement. With shaft 214 anchored in sacrum S and washer 376 disposed in engagement with outer non-articular surface NA, further rotation of screw 360 relative to shaft 214, axially translates shaft 214 relative to screw 360 in a configuration to draw separated articular surfaces A of sacrum S and ilium I into fixation to immobilize sacro-iliac joint J, similar to the non-compression and compression orientations described above.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A sacro-iliac implant comprising:
an inner member including an inner surface and extending between a first end and a second end configured for penetrating a sacrum;
an outer member extending between a first end configured to engage an outer non-articular surface of an ilium and a second end; and
an actuator disposed with the outer member and defining an outer surface,
wherein the actuator is movable relative to the outer member such that the outer surface of the actuator engages the inner surface to cause axial translation of the inner member relative to the outer member in a configuration such that separated articular surfaces of the sacrum and the ilium are drawn into fixation.

2. A sacro-iliac implant of claim 1, wherein the inner member slidably engages the outer member during the axial translation.

3. A sacro-iliac implant of claim 1, wherein the inner member includes a substantially even outer surface and the outer member includes a substantially even inner surface such that the inner member slideably engages the outer member during axial translation.

4. A sacro-iliac implant of claim 1, wherein the outer member includes an outer surface that defines a first diameter portion and a second greater diameter portion adjacent the first end of the outer member, the second diameter portion including a surface configured to engage the outer surface of the ilium.

5. A sacro-iliac implant of claim 1, wherein the first end of the outer member includes a flange that defines a cavity configured for disposal of the actuator.

6. A sacro-iliac implant of claim 1, wherein the first end of the outer member defines an end surface and the actuator includes a first end defining an end surface, the end surfaces being disposed in substantial alignment.

7. A sacro-iliac implant of claim 1, wherein the actuator includes a first end defining a tool socket and a second end defining a threaded outer surface.

8. A sacro-iliac implant of claim 1, wherein the inner surface of the inner member is threaded adjacent the first end and substantially even adjacent the second end.

9. A sacro-iliac implant of claim 1, wherein the inner member includes an outer surface that defines a first diameter portion and a second greater diameter portion adjacent the second end.

10. A sacro-iliac implant of claim 1, wherein the inner surface of the inner member defines a passageway having a first diameter adjacent the first end and a second smaller diameter adjacent the second end.

11. A sacro-iliac implant of claim 1, wherein the actuator is rotatable relative to the outer member and the inner member to cause the axial translation.

12. A sacro-iliac implant system of claim 1, wherein the first end of the outer member includes at least one fixation element oriented to penetrate the outer surface of the ilium.

13. A sacro-iliac implant comprising:
an inner member including an inner surface and a substantially even outer surface, the inner member extending between a first end and a second end configured for penetrating a sacrum;
an outer member extending between a first end configured to engage an outer non-articular surface of an ilium and including a flange that defines a cavity and a second end, the outer member including a substantially even inner surface, the first end of the outer member defining an end surface; and
an actuator disposed with the flange cavity and defining an outer surface, the actuator including a first end defining an end surface, the end surfaces being disposed in substantial alignment,
wherein the actuator is rotatable relative to the outer member and the inner member such that the outer surface of the actuator engages the inner surface of the inner member to cause axial translation of the inner member relative to the outer member in a configuration such that separated articular surfaces of the sacrum and the ilium are drawn into fixation.

14. A sacro-iliac implant system of claim 13, wherein the second end of the outer member includes a first transverse flange configured to engage the outer surface of the ilium and a second axial flange that defines a cavity configured for disposal of the actuator.

15. A sacro-iliac implant system of claim 13, wherein the actuator includes a screw having a head defining a tool socket and a threaded shaft.

16. A sacro-iliac implant system of claim 13, wherein the outer member includes a sleeve defining an interior passageway configured for disposal of the inner member.

17. A sacro-iliac implant system of claim 13, wherein the inner member includes a cannulated shaft configured for disposal of the actuator.

18. A sacro-iliac implant system of claim 13, wherein the inner surface of the inner member defines a passageway having a first diameter adjacent the first end and a second smaller diameter adjacent the second end.

19. A sacro-iliac implant system of claim 13, wherein the first end of the outer member includes at least one fixation element oriented to penetrate the outer surface of the ilium.

20. A sacro-iliac implant comprising:
an inner member including an inner surface and an outer surface, the inner member extending between a first end and a second end configured for penetrating a sacrum; and
an outer member extending between a first end including a flange and being configured to engage an outer non-articular surface of an ilium and a second end,
wherein the inner member is rotatable relative to the outer member such that the outer surface of the inner member adjacent its first end engages the outer member to cause axial translation of the inner member relative to the outer member in a configuration such that separated articular surfaces of the sacrum and the ilium are drawn into fixation.
